(19) United States
(12) Patent Application Publication
(10) Pub. No.: US 2010/0047294 A1
(43) Pub. Date: Feb. 25, 2010

(54) SEMI-FINISHED PRODUCT IN THE MANUFACTURE OF A COMPOSITION FOR SKIN OR HAIR CARE

(75) Inventor: Thomas Ahlnas, Kotka (FI)

Correspondence Address: YOUNG & THOMPSON, 209 Madison Street, Suite 500, Alexandria, VA, 22314, US

(73) Assignee: OY GRANULA AB LTD, Kotka (FI)

(21) Appl. No.: 12/377,515

(22) PCT Filed: Aug. 14, 2007
(86) PCT No.: PCT/FI07/00201

§ 371 Date: May 25, 2009

(30) Foreign Application Priority Data
Aug. 14, 2006 (FI) 20060729

(51) Int. Cl. A61K 9/10; A61K 9/107; A61K 36/13; A61K 8/18; A61Q 1/06; A61Q 17/04

(52) U.S. Cl. 424/401; 424/484; 424/770; 424/64; 424/59

(57) ABSTRACT

A semi-finished product is manufactured from raw liquid, extract or powder obtained from wood material, an end product composition for skin or hair care being obtained from the semi-finished product by adding one or more components thereto. The semi-finished product optionally contains a UV protector, and is a liquid, emulsion, microemulsion or nanoemulsion or a dispersion, suspension, wax, lotion or gel or a solid substance. The semi-finished product further contains an active ingredient mixture which contains phenolic compounds having antioxidative and/or microbicidal properties and/or abilities to capture free radicals. The active ingredient mixture contains as phenolic compounds at least one oligolignan, and at least two lignans, tannins or flavonoids selected from a group consisting of hydroxymatairesinol, matairesinol, oxomatairesinol, lariciresinol, cyclolariciresinol, secoisolariciresinol nortrachelogenin, pinoresinol, pinosylvine, conidendrine, melacasidine, teracasidine, ellaga-tannin or a reaction product, stereoisomer or derivative of these compounds.

The invention relates to the semi-finished product according to the preamble of Claim 1.

Lignans and flavonoids are natural antioxidants, which are obtained from wood material and which are also capable of capturing the energy-intensive free radicals that are generated by the photo-exposure of various compounds. Having effects similar to estrogen on human and animal cells, lignans and flavonoids are phenolic compounds, phytoestrogens. Therefore, lignans have been recommended to be used, among others, in the prevention of cancer, and in treating certain hormonal and cardiovascular diseases.

Lignans and some flavonoids are widely found in wood materials, especially in knot wood and branches, whereby their availability is so good that they can be considered to be used in large quantities in other industrial sectors as well. The antioxidative capacity and the ability of lignans to capture free radicals depend on their degree of glycosylation; generally, lignans are found as aglycols in wood material. In addition to lignans, there are also other phenolic compounds in wood, such as tannins, oligolignans, flavonoids, stilbenes.

Even though the general availability of the lignans and flavonoids found in wood is good, it has been difficult to isolate and further use them in dermatologic compounds, as the well-known isolation methods adapted to lignans have been based on extracting the wood material, especially chopped spruce material, with various extraction methods based on acetone. The desired individual lignan compounds, such as hydroxymatairesinol, (HMR), matairesinol, pinoresinol, etc., have then been precipitated from the lignan extracts obtained, and they have been further processed to manufacture various cosmetic preparations and drugs for skin care (cf., e.g., WO 2004/000304). A lignan extract thus isolated is not suitable for skin and hair care products because of the smell of acetone and the cytotoxicity, which is why complex methods must have been used for the purification of lignans to remove the acetone from the extract. Hence, the isolation, purification and selection of lignans for each use have been relatively laborious and time-consuming. Alternatively, lignans have been prepared by the methods of organic chemistry by synthesizing, but then the price of the product easily becomes too high.

Among others, published applications WO 00/13661 and US 2005/0169947 suggest that wood-based lignans be used in dermatologic compositions. However, the methods of isolating and manufacturing lignans that are described in these publications are based on the processes described above.

Based on the prior art mentioned above, the aim of the applicant was to simplify the isolating process of lignans and other phenolic compounds, phytoestrogens, obtained from wood, from the wood material, and to provide a product containing phytoestrogens as active ingredients, which is suitable to various needs of the cosmetic industry. Another object was to provide new methods of application for the phytoestrogens isolated from wood material.

The invention relates to the semi-finished product according to Claim 1. To be more precise, the invention relates to the semi-finished product, which is prepared from the raw liquid, raw extract or raw powder obtained from the wood material, the end product composition for skin or hair care being obtained from the semi-finished product by adding one or more components thereto. In that case
the semi-finished product optionally contains a UV protector,
the semi-finished product is a liquid, such as solution, emulsion, microemulsion or nanoemulsion, or a semi-solid substance, such as dispersion, suspension, wax, lotion or gel, or a solid substance, and the skin or hair care composition (the end product) can be prepared from the semi-finished product by adding liquid thereto,
the semi-finished product contains an active ingredient mixture, which is obtained from wood material or bark by grinding, extracting or pressing and which contains phenolic compounds obtained from the wood material, the compounds having antioxidative and/or microbicidal properties and/or abilities to capture free radicals,
the active ingredient mixture contains at least one oligolignan as phenolic compounds,
in addition, the active ingredient mixture contains, as phenolic compounds, at least two lignans, tannins or flavonoids, which are selected from a group consisting of hydroxymatairesinol, matairesinol, oxomatairesinol, lariciresinol, cyclolariciresinol, secoisolariciresinol, nortrachelogenin, pinoresinol, pinosylvine, conidendrin, melacasidine, teracasidine, ellagatannin or a reaction product, stereoisomer or derivative of these compounds.

The active ingredient mixture herein refers to a liquid mixture, which is obtained from the wood material, generally, by grinding, extracting or pressing and which comprises oligolignans, tannins, lignans and flavonoids originating in the wood material.

The raw liquid and the raw extract refer to the active ingredient mixture and a liquid obtained from the wood material, possibly containing a solvent.

The raw powder refers to the active ingredient mixture and wood powder that comprises ground wood material.

The semi-finished product in this context refers to a composition containing a liquid, semi-solid or solid active ingredient mixture, from which the end-product composition (is obtained) by adding one or more components thereto, at least one of them being a liquid component. The semi-finished product is often a concentrate, such as a raw extract concentrate, to which a 7 to 20-fold amount of one or more solvents are added to prepare the end product.

The active ingredient mixture is preferably obtained by grinding or extracting the wood material of at least two different wood species, which is obtained from knot wood, branches or bark. The active ingredient mixture is especially preferably obtained from knot wood pulp.

Differing from the previous practice in the art, the invention is firstly based on the fact that the raw extracts, raw liquids and raw powders that are recovered from the wood material, such as wood chips or chipped wood pulp of the chemical pulp industry, which contain lignans and/or flavonoids and/or tannins as active ingredients, are either used as such or only slightly purified for the manufacture of the semi-finished products of the skin or hair care compositions. This provides the advantage that the isolation of the raw product from the wood material is simple and requires a considerably smaller number of process stages than before. The raw extract is preferably a mixture that originates in two or more wood species, such as spruce and pine or conifer and birch. When pulp containing knot wood is separated from the pulp line, in practice, it is impossible to economically separate clean knot wood pulp of spruce, but in Finland, it always contains pine wood and, correspondingly, pine knot wood pulp always contains some spruce. The types and amounts of lignans obtained from different wood species vary considerably. Furthermore, other compounds advantageous for the end product are obtained from other wood species by grinding or extracting, such as microbicidal compounds (pine) or substances for treating illnesses or to be used as microbicides (birch, betulinic acid). By extracting or grinding a mixture of different types of wood, active ingredient mixtures are obtained having different properties, such as antioxidative, microbicidal and, possibly, disease-preventive, and abilities to catch free radicals.

Secondly, the invention is based on the fact that the phenolic compounds, such as lignans and flavonoids contained in the wood material, are extracted from the wood with an extraction medium, which as such is suitable for various cosmetic products. Previously, acetone-based solvents have been used for the extraction to facilitate the further purification of the extracted lignan and/or flavonoid mixture. However, as the purpose of the invention is to use the extract or the material obtained from grinding as such in the manufacture of various semi-finished products and end products, the solvent used in the extraction can simply be selected on the basis of its suitability to further use.

In that case, suitable media for the extraction of wood include various alcohol-based solvents, such as butylene glycol, butylene glycol+monoalcanol, glycerol or glycerol+alcohol, which are widely used in the cosmetic industry. The extraction solution preferably contains a lower monovalent or bivalent alkyl alcohol or a mixture of the lower monovalent and bivalent alkyl alcohols and/or glycerol, or a mixture of the lower (monovalent) alkyl alcohol and the bivalent or a trivalent lower alkyl alcohol. The mixture of the lower alkyl alcohol and glycerol or alkyl glycol is a preferable solvent medium, as the need to further process the raw extracts (=solvent+active ingredient mixture) or raw extract concentrates based on extraction solutions containing such alcohols is as small as possible, when preparing different end compositions and/or their semi-finished products. The alcohol-based raw extract or raw extract concentrate is well-suited to the manufacture of skin or hair care compositions, as many products in the art, such as fragrances, shampoos, moisture emulsions and lotions are based on the alcohol-based homogeneous solutions and dispersions.

The monovalent alcohol is preferably ethanol, propanol, butanol, heptanol, octanol or decanol. The monoalcohol is especially preferably ethanol.

The bivalent alkyl alcohol is preferably a lower alkylene glycol, which is preferably selected from a group comprising ethylene glycol, propylene glycol, butylene glycol, pentylene glycol, and dipropylene glycol. The latter is especially preferable to be used in fragrances. Butylene glycol is an especially preferable solvent, as the lignan and/or flavonoid compounds according to the invention dissolve therein in amounts of over 10% and it is physiologically tolerable. Solutions of ethanol and bivalent alcohols are preferable for dissolving and also extracting the lignan and flavonoid mixtures according to the invention, whereby the bivalent alcohol is especially preferably ethylene glycol, propylene glycol, butylene glycol, pentylene glycol, dipropylene glycol. When the raw extract is formed from the active ingredient mixture and glycol, or glycol+alcohol, the problem of migration/absorption of the active ingredient mixture is solved in the end product made of the raw extract: when the end product is spread onto the skin, the water/alcohol contained in the solvent system evaporates quickly and the active ingredient mixture can remain/deposit on the skin. Glycol does not evaporate but dissolves the raw extract well, enhancing its migration into the skin.

Suitable trivalent alcohol solvents are glycerols.

Thirdly, the invention is based on the fact that the selection of the lignans and/or flavonoids incorporated into the raw product is rather based on their properties to capture free radicals than the antioxidative properties that prevent the breakage of double bonds. The selection of the lignan or flavonoid compounds incorporated in the semifinished and end products of the prior art has primarily been based on their antioxidative properties. Generally, the antioxidative properties of a certain lignan or flavonoid distinctly differ from its capacity to capture free radicals. Free radicals are generated, when UV light induces the energy-intensive derivatives of a substance. The lignan or flavonoid mixtures that have the capacity to capture free radicals can be used in the active ingredient mixtures in previously unknown uses.

Fourthly, the invention is based on the fact that the raw liquid or raw extract according to the invention is harmless, non-cytotoxic to the organism, whereby it can be used in large enough amounts (as much as 1 to 3% of the weight of the composition) to prevent the adverse effects of the free radicals on the organism. Many prior art compounds that are intended for this purpose, such as BHT or BHA, are cytotoxic, whereby their amounts available are too small (about 0.1% by weight of the composition at the most) for them to be efficient enough in capturing the free radicals on the skin.

Fifth, the invention is based on the fact that the composition of the active ingredient mixture (oligolignan, the lignan, flavonoid, and tannin compounds) the quality and quantity with respect to each other) contained in the cosmetic composition and the relative amount of the active ingredient mixture in the composition are selected on the basis of the properties of the components contained in the active ingredient mixture to capture free radicals, their possible microbicidal properties and the properties of migration into the organism/body required of the active ingredient. The composition of the active ingredient mixture is selected on the basis of the use, the isolation method, and the type(s) of wood available, from which the active ingredient mixture is ground, pressed or extracted. The amount and composition of the active ingredient mixture in the cosmetic end product depend on the properties to capture free radicals/the antioxidative properties required at each time, and the application. The body contains various polar, semi-polar, and non-polar parts, or gans and mebranes, which is why the active ingredient mixture should also contain oligolignans, lignans, tannins, and flavonoid compounds, which have different capturing mechanisms of free radicals and migration properties. Examples of the various objects of the body, which require active ingredients that have different capturing mechanisms of free radicals and different migration: cell wall (semi-polar), the exterior of the cell, water phase (polar), and hydrophobic (the body parts that contain fat). For example, if only one purified lignan compound was used as the active ingredient in the composition, its migration and action in the different places of the body would be considerably weaker than those of the active ingredient mixture according to the invention.

The skin or hair care composition or the semi-finished product used in the manufacture thereof preferably contains oligolignans and at least two different lignans that are selected from a group consisting of hydroxymatairesinol, matairesinol, oxomatairesinol, lariciresinol, cyclolariciresinol, secoisolariciresinol, nortrachelogenin, pinoresinol, pinosylvine, conidendrin, melacasidine, teracasidine, ellagatannin, or a reaction product, derivative or stereoisomer of these compounds.

The composition or the semi-finished product preferably contains at least two different lignans, which are selected from a group consisting of hydroxymatairesinol, matairesinol, oxomatairesinol, cyclolariciresinol, nortrachelogenin, pinoresinol, pinosylvine, secoisolariciresinol, or a reaction product, stereoisomer or derivative of these compounds.

In this context, the derivatives and the reaction products refer to the modifications that either occur naturally in the wood material or are generated, when isolating (from the said lignans, flavonoids and tannins) the raw products according to the invention or processing the semi-finished products or the end products. Natural modifications of lignans include, among others, partly or fully glycocided lignans and the aglycans of lignans. The derivatives and reaction products most important to lignans are: for hydroxymatairesinol: dimethylmatairesinol, 7-methoxymatairesinol, didemethylmatairesinol, isohydroxymatairesinol, oxomatairesinol, enterolactone, conidendrin, 7′,8′-dehydro-7-hydroxymatairesinol; for secolariciresinol and lariciresinol: cyclolariciresinol, 7-hydroxysecoisolariciresinol; for the pinosylvine common in pines: the monoether of pinosylvine, such as monomethylether and dihydropinosylvine and the monomethylester of dihydrosylvine; for pinoresinol: dimethylpinoresinol; for nortrachelogenin: liovil. In connection with Example 1, further examples of the derivatives of these lignans are given, which are considered to fall within the scope of the invention.

Previously, in dermatologic compositions (cf. US 2005/0 169 947), certain pure lignans have been suggested to be used because of their antioxidative properties. In particular, the reference in question cites the use of purified hydroxymatairesinol or a derivative thereof in dermatological applications, based on the antioxidative effect thereof. When lignan or flavonoid is used in a dermatological composition because of its antioxidative effect, its range of use is considerably more limited compared to when primarily taking into consideration the ability of the compound to capture free radicals. Furthermore, the use of purified lignans in dermatological compositions has the disadvantage that they do not act in all parts of the body as desired, contrary to the active ingredient mixtures containing oligolignans according to the invention. It is described hereinafter, how taking into account the ability of the compound to capture free radicals has a considerably positive effect on the scale of the range of use. Furthermore, it has been observed that some active ingredient mixtures according to the invention also show antimicrobial effects, which further increases their utilization rates.

The raw products according to the invention have been observed to provide considerable additional advantages earlier unknown:

Surprisingly, it has been observed that in the end products manufactured from the liquid concentrates that contain the active ingredient mixtures, which have been extracted from wood material by the method according to the invention, the mixtures are more effective than individual pure lignans, flavonoids or tannins.

Furthermore, there are oligolignans originating in the wood material always present in the active ingredient mixture of the semi-finished products according to the invention, which is quite new in the art. The solubility/migration properties of oligolignans depend on the polarity of the solvent system and they are different from those of corresponding lignans, which is of advantage in several cosmetic compositions. For example, in a composition containing a glycol/alcohol solvent system, the oligolignans remain well in place when the water evaporates, which promotes their penetration into the skin. Oligolignans have similar antioxidative properties and abilities to capture free radicals as corresponding monomers do.

The raw extract according to the invention is preferably an HMR raw liquid, its active ingredient mixture that has been extracted from wood material mainly consisting of (by 60 to 70%) hydroxymatairesinol, matairesinol and/or oxomatairesinol, or the isomeric forms thereof. The raw extract also contains 5 to 30% of oligolignans. Oxomatairesinol is generated, if an extraction process is used, wherein the extraction solution is heated to about 200° C. Oxomatairesinol has been discovered to have an ability to capture free radicals comparable to that of hydroxymatairesinol or even better.

The use of the various forms of HMR raw liquid (HMR raw extract, HMR raw liquid) according to the invention in the manufacture of liquid concentrates, which are used as semi-finished products, and of also cosmetic compositions is quite new in the art. Such semi-finished products according to the invention comprise the components of the skin care and hair care products that have been blended with the HMR raw product or its derivatives or their oligomers.

The semi-finished product according to the invention can preferably (be used) in the skin or hair care compositions, which require the function to capture free radicals, which are a generated by the UV light of the sun. Previously, products containing pure lignans have been proposed to be used to protect the skin against the aging effect of light mainly as antioxidants (US 2005/0169947). Instead, the cosmetic preparations according to the invention, which contain tannin, lignan and flavonoid mixtures as active ingredients and provide protection against sunlight, as described in detail below, and the liquid concentrates used in the manufacture thereof are quite new in the art and inventive as such. These preparations can be used, among others, in sun protection products, such as lotions, sprays, and moisture emulsions that contain substances, which protect against the UV light of the sun, to enhance the effect of the substances that are used as UVA and UVB filters of sunlight, but to also capture the free radicals, which are possibly released from the substances used as the UV filters and which otherwise might damage the skin.

A special application of the latter use comprises the use of the raw extract obtained from wood material or the raw liquid pressed from wood in protecting the coating of the physical UV filter particles that are coated with silica compounds or aluminium.

If a sufficient amount of liquid concentrate according to the invention is added to the sun protection composition in connection with the manufacture, actual UV filter media are not necessarily needed in the composition, as the active ingredients originating in the raw product prevent the adverse effects of sunlight on the skin. Such use has not been possible earlier, as there has been no source of lignan and/or flavonoid available, which would have been inexpensive and physiologically well-tolerated in large amounts, and which could easily be added to sun protection compositions.

Another quite new range of use comprises the use of the raw products according to the invention, or their derivatives, in protecting the hair against the effects of sunlight that fade and stress the hair.

One important new range of use of the raw product according to the invention comprises various compositions intended for the treatment or prevention of exposure to heat, such as cooling moisture emulsions and skin creams, compositions for shaving, such as balms and after-shave lotions, and skin surgical compositions for protecting scar tissue. In these compositions, the tannin, lignan and/or flavonoid mixtures obtained from the semi-finished products according to the invention are used in capturing the free radicals that are induced from the components of the composition onto the skin or inside the skin under the effect of heat, before the said radicals damage the skin.

Another new range of use comprises blending the raw powders, raw liquids, and raw extracts for the compositions of colour cosmetics. Ingredients may come off the compositions of colour cosmetics (e.g., lipsticks and lipsalves) which, when entering the organism, develop free radicals under the effect of UV light, damaging the organs. The active ingrediens originating in the semi-finished product according to the invention prevent the free radicals, which have possibly been generated by the ingredient that came off the composition, from damaging the organism.

When the semi-finished product according to the invention is mixed with compositions containing substances like melanin, the active ingredients contained in the semi-finished product prevent the adverse effects of the ionising UV radiation of sunlight on the skin. Substances like melanin herein refer to biopolymers similar to the melanin obtained from nature and synthetic melanins, which have essentially the same capacity of heat, radiation and light transmission as melanin does. Due to the expensiveness of natural melanin, substances similar to melanins are used instead, which generally include antioxidative effects and/or oligolignan components that capture free radicals, colouring agents, and UV filters. By using the raw products according to the invention in the manufacture of compounds similar to melanin, some or all synthetic antioxidative components can be totally omitted.

The raw extract or the raw extract concentrate obtained from the wood material is preferably obtained by extracting the wood material obtained from knot wood with a physiologically acceptable solvent, such as a lower polar organing solvent, and thereafter by possibly concentrating the extract solution containing the lignan and/or flavonoid mixture. Preferable polar organic extract solutions include the alcohol-based extract solutions previously mentioned. So-called ancat solutions can also be used for the extraction.

The semi-finished liquid contains over 5%, preferably over 7%, and most preferably 10% by weight of the active ingredient mixture, i.e., components originating in the wood material. The amount of active ingredient mixture in the end products is 0.1 to 3%, more preferably 0.5 to 3% by weight. The raw extracts or raw extract concentrates containing the active ingredient mixtures extracted from the wood material are often usable semi-finished products as such. The preferable raw extract concentrates according to the invention that are used as semi-finished products include concentrated raw extracts, which contain a tannin, lignan and/or flavonoid mixture in a dissolved form and, in addition, as a solvent: lower alkyl glycols, such as glycerine, propylene glycol, butylene glycol, pentylene glycol, and dipropylene glycol.

The preferable raw extract concentrates according to the invention, which are used as semi-finished products, further include those containing an HMR lignan mixture in a dissolved form and, in addition, the above-mentioned lower monoalcohols as solvent. The HMR lignan mixture mainly (by 60 to 70%) contains hydroxymatairesinol and/or matairesinol and/or oxomatairesinol, secolariciresinol, lariciresinol obtained from spruce, or a reaction product, derivative, diastereomer or a pure isomer thereof. In addition, the mixture contains smaller amounts of other lignans, flavonoids and oligolignans.

The preferable raw extract concentrates according to the invention, which are used as semi-finished products, further include those containing a lignan and/or flavonoid mixture (=the active ingredient mixture) in a dissolved form and also, as the solvent, a mixture of lower alkyl glycols and lower (mono)alcohols, such as ethanol, together with glycerol, propylene glycol, butylene glycol, pentylene glycol or dipropylene glycol. Of these, ethanol together with glycol is especially preferable, as glycol is a moisturizer used in several skin care compositions and, in addition, it has an antibacterial effect, whereby such a raw extract concentrate that is used as the semi-finished product is well-suited to the manufacture of deodorants, for example.

The semi-finished product can also be raw powder or raw liquid. The raw powder contains ground knot wood and the raw liquid contains liquid obtained from the wood material by pressing. The raw product can also be isolated from the wood material by other extraction methods well-known in the prior art; regarding them, a reference is made to the literature in the field.

Typical components of skin and hair care compositions that are obtained from the semi-finished products comprise fragrances and the solutions, emulsions, microemulsions, dispersions, suspensions, gels, foams, aerosols and sprays that are used as the base material for the compositions.

A preferred embodiment of the invention relates to liquid semi-finished products that contain the raw extract and often also include stabilizing agents, such as emulsifiers, which are needed in the homogenization of the dispersion/suspension in further processes. The emulsifier is preferably an amphoteric (zwitterionic) surfactant. The amphoteric surfactant in this application refers both to individual compounds that have anionic and kationic groups in the same molecule, and also to amphoteric-like surfactants, which are formed from an anionic and a kationic organic component, as described in patent specification U.S. Pat. No. 5,811,082. In particular, short-chain amphoteric-like substances, such as the solution of ethanolamine and lower alkyl alcohol (so-called ancat solution), can also be used as a preferable extraction medium. Hereinafter, examples of preferable amphoteric and amphoteric-like substances are given.

The liquid semi-finished products, which are used as the base material for the end product compositions that contain UV-protectors, comprise homogeneous semi-finished solution products, liquid-liquid suspension semi-finished products, liquid-liquid dispersion semi-finished products, liquid-liquid microemulsion semi-finished products or liquid-liquid nanoemulsion semi-finished products. These semi-finished products are based on the polar and/or non-polar organic solvents or solvent mixtures, which contain the oligolignan, tannin, lignan and/or flavonoid mixture according to the invention and which are used in forming homogeneous solutions, or dispersions or suspensions containing two or more liquid phases as mixtures.

The homogeneous semi-finished solution products according to the invention contain non-polar or preferably polar solvents or solvent mixtures, which contain the oligolignan, tannin, lignan and/or flavonoid mixture (the active ingredient mixture) according to the invention, originating in wood material. The semi-finished solution products according to the invention are preferably based on organic protic polar solvents, such as monovalent alcohols and/or bivalent alcohols and/or trivalent alcohols or the mixtures thereof, the lignan and/or flavonoid mixture according to the invention being dissolved therein. The organic protic polar solvents preferably comprise the solvents mentioned earlier, which have been used for extracting the tannin, lignan and/or flavonoid mixture from the wood material, as they considerably simplify the manufacture of the semi-finished products.

The liquid semi-finished products according to the invention can be liquid-liquid emulsion dispersions, liquid-liquid microemulsions or liquid-liquid emulsion suspensions, whereby one of the liquid components includes the raw extract or the raw extract concentrate, or all of it consists of such raw extract or raw extract concentrate. The semi-finished products according to the invention can also be multiphase emulsions consisting of several liquid phases, their aqueous phase containing the active ingredient mixture according to the invention. The raw extract or the raw extract concentrate is preferably a solution including a polar solvent and the tannin, lignan or flavonoid mixture according to the invention. The polar solution is preferably an alcohol solution, a mixture of a monovalent and a bivalent alcohol or an aqueous/alcohol solution, which are listed above. In the manufacture of the liquid-liquid emulsion dispersions, liquid-liquid emulsion suspension, liquid-liquid microemulsions and microemulsions according to the invention, which are used as the semi-finished products, various emulsifiers or mixtures or different emulsifiers are used, which are presented, for example, in References 1 ja 2 and the INCI list (International Nomenclature of Cosmetic Ingredients) 4 and the corresponding list 5 of cosmetic substances, approved by the FDA.

To promote the usability of the said W/O or O/W emulsions and microemulsions, which are used as semi-finished products, in different skin and hair care compositions, suitable surfactants that comprise dispergents or mixtures of dispergents are often added to the semi-finished products in advance. The quality and the quantity of these surfactants depend on the end use of the semi-finished product. Suitable surfactants for the manufacture of cosmetic emulsions, gels, foams, and aerosols are described, among others, in References 1 and 2 and the INCI list (International Nomenclature of Cosmetic Ingredients) 4 and the corresponding list 5 of cosmetic agents approved by the FDA.

Particularly suitable surfactants include lecithin and its derivatives, long-chain alkyl amines, and alkyl ammonium compounds, polyethoxylated amines and pyridium salts, capryl/caprine mono/diglyceride, a mixture of alkyl glycoside and alkyl alcohol, polyglycerol-3-di-isostearate and polyglycerol+dihydroxy stearate, such as Lameform TGI/Dehumuls PGPH, cetyl polyglyceride PEG/PPI dimethicone, polyglycerol-4-isostearate+cetyl dimethicone copolyol, silicone-based emulsifiers, polyglycerol isostearate+polyglyceryl-3-methyl glucose distearate.

If the purpose is to manufacture, from the liquid semi-finished products containing the active ingredient mixture according to the invention, e.g., semi-solid lotion compositions, such as sun protection creams that are spread onto the skin, the semi-finished products are mostly water-oil emulsion dispersions that contain an oil phase as a continuous phase, wherein the water-alcohol phase is dispersed in the continuous oil phase, or oil-water dispersions, wherein the oily phase is dispersed in the continuous water-alcohol solution. Possible liquid semi-finished products also include microemulsions, wherein drops of water or lipid of the micro or nano size have been incorporated into the continuous liquid phase by dispersion.

Some important semi-finished products according to the invention are obtained by extracting the wood material with so-called “ancat” substances, which are organic salts that behave amphoterically (zwitterionically), their formation being described in detail in patent specification U.S. Pat. No. 5,811,082, or solutions, which are formed from an acidic and an alkaline organic solution and have buffering properties (e.g., a solution of ethanol amine and formic acid). Such solutions that contain the ancat compounds are well-suited to the extraction of the lignan and/or flavonoid mixtures according to the invention from knot wood. In the raw extracts or raw extract concentrates thus obtained, the amphoteric-like ancat emulsifier or ancat dispersant contained in the solution contributes to rendering the solution W/O-based or O/Wbased semi-finished emulsions, and/or when manufacturing cosmetic end products, such as sun protection products (cf. Example 5) either from the semi-finished products or directly from the raw extracts or raw extrac concentrates. The ancat emulsifiers and dispersants can also be added to the W/O or O/W emusions and microemulsions ang gels, which are used as the semi-finished products, to enhance their usability, when further manufacturing the cosmetic end products.

If the liquid semi-finished products according to the invention are intended for sun protection products, they often also include UV filter media, such as physical UV filter media, among others, TiO₂, CeO₂, ZnO and/or organic UV filter media, such as MBBT. In that case, they generally also include dispersants, which advance the mixing of solid particles with the liquids and which are described in detail in the literature in the field, such as the previously-mentioned INCI list 4.

The invention further relates to the gel-like semi-finished products used in the manufacture of the skin and hair care compositions. These, in turn, are either manufactured from raw powder, a suitable liquid being dispersed therein, or raw extract, raw extract concentrate or raw liquid, particles of a solid substance being dispersed therein. The anionic, kationic and amphoteric dispersants used in the manufacture of the gel-like semi-finished products are well-known in the art, e.g., from the INCI list 4. The above-mentioned amphoteric-like ancat dispersants can also be used, when producing the semi-finished products in question. The ancat dispersants can also be added to the above-mentioned semi-finished products to enhance their processability in making the end products (cf. Example 5).

The gel-like semi-finished products are primarily used in the manufacture of gel-like sun protection compositions that are spread onto the skin or shampoo-like compositions that provide protection against UV light, which is why UV protectors are usually added thereto, being dealt with hereinafter. Furthermore, dispersants that are used in the manufacture of the end product can be added thereto in advance; reference being made to the literature in the field, such as 4, 5.

The liquid and gel-like semi-finished products also often contain, when finished, other additives generally used in skin and hair care end product compositions, suahd as moisturizers, moisture-absorbing agents, softeners, greases, lubricants, fragrances, thickening agents, microbicides, thickeners, and antioxidants (other than the antioxidative components originating in the lignan and/or flavonoid mixtures according to the invention).

The invention further relates to the semi-finished fragrance products used in the manufacture of the skin and hair care compositions. Those include fragrances, which are blended with the raw products, such as the raw extracts, raw liquids or raw powders and used in the art, cf. 4 and 5. The fragrances are preferably selected from a group consisting of phenyl ethyl glycol, eugenol, isoeugenol, geraniol, citronellol or linalol, or their esterified forms or their aldehydes.

The liquid end product composisitons obtained from the semi-finished products according to the invention can be formulated, among others, into liniments and moisture emulsions, which are homogeneous solution compositions, liquid-liquid emulsion compositions or liquid-liquid microemulsion compositions with a low viscosity. These are either manufactured from the liquid semi-finished products according to the invention, which were dealt with above or, alternatively, starting with solution-like raw extract concentrates or raw extracts, and by blending them according to the conventional procedures in the art to form homogeneous end products. The dispersants used in the manufacture of the liquid end product compositions are much the same as those described in connection with the manufacture of the semi-finished products, or they are otherwise well-known in the art 4, 5.

Examples of the liquid end product compositions include after-shave products and moisturizing and cooling emulsions, which are used after sunbathing (after-sun products) and, generally, after a thermal stress on the skin. These compositions need no UV filter media, and the active ingredient mixtures according to the invention, when used therein, capture the free radicals that are possibly generated from the product in the thermal stress (preventing them) from penetrating deeper into the skin layers. Examples of these compositions are given hereinafter.

The semi-solid end product compositions containing the lignan or flavonoid mixture or its derivative can be formulated into viscous creams and gels, for example. These can be liquid-liquid dispersion compositions or liquid-liquid microemulsion dispersion compositions, liquid-liquid suspension composition, liquid-solid dispersions or liquid-solid suspensions, which are homogenized by a method conventional in the art. The gel-like end product compositions according to the invention include, e.g., shampoos and creams intended as sun protection agents, and lipsticks and lipsalves, which contain the active ingredient mixture according to the invention, and, possibly, agents intended as UV filters. The semi-solid end product compositions also include products containing various colouring agents used in cosmetics, such as lipsticks and creams containing an agent similar to melanin, whereby the purpose is to prevent the free radicals induced from the colouring agents by light from damaging the skin or the organism. The semi-solid end product compositions are either manufactured from the liquid or solid semi-finished products according to the invention, which were dealt with above or, alternatively, starting with the raw extract concentrates, raw extracts, raw liquids or raw powder, by blending them according to the conventional procedures in the art to form homogeneous end products.

If these liquid and semi-solid compositions, which contain the active ingredient mixture according to the invention that originates in the lignan and/or flavoinoid mixture obtained from the wood material, or a derivative thereof, are intended as sun protection compositions, they mostly include physical and/or organic UV filter media.

The invention further relates to semi-finished products that are intended for forms of aerosols, such as sprays. The aerosol compositions comprise dispersions, wherein the solid matter or liquid is dispersed into a very fine form in a continuous gas phase. In the invention, the solution or solid matter is dispersed in the gas phase of the aerosol, containing the active ingredient mixture originating in the semi-finished product. Generally, the active ingredient mixture of the liquid phase is obtained from the raw extract, raw extract concentrate or raw liquid, which are possibly mixed with a suitable solvent or dispersed in another liquid, such as oil or lipid. The solid matter either comprises pure raw powder or raw powder, which suitable additives have been added to. Liquid and solid aerosols can also be manufatured from the solid and liquid semi-finished products according to the invention, which are described above.

One important application of the aerosols according to the invention comprises protecting the hair against the fading effect of sunlight. In that case, the aerosol composition generally contains physical or organic UV protectors and the active ingredient mixture originating in the semi-finished products according to the invention. Examples of substances that protect against the UV light of the sun include physical UV filter media or organic filter media that absorb UV light. These include, among others, TiO₂, FeₓO_{y}, ZnO, MBBT (methylene-bis-benzotriazolyl tetramethyl butyl phenol), EHMC (ethyl methoxycinnamate), BNBM (ethyl hexyl methoxy dibenzoyle methane) BMC (4-methyl benzylidene camphor), BP3 (3-benzophenone), 5-benzophenone, DTS (drometrizole trisiloxane), DPDT (disodium phenyl dibenzimidazole tetrasulphone), BEMT (bis-ethyl oxyphenol methoxyphenyl triazine), IMC (isoamyl-p-methoxycinnamate) PBSA (phenyl benzimidazole sulphonic acid), OT (octyl trazone), OS (octyl salicylate), TDSA (terephthalidene dicamphor sulphonic acid), Parsol Slx (benzylidene malonate polysiloxane).

The invention also relates to the solid, liquid and semi-solid compositions for skin or hair care, which contain an active ingredient mixture originating in the tannin, lignan and/or flavonoid mixture obtained from the wood material, or a derivative thereof. The lignan and/or flavonoid mixture is preferably used as such (without isolation) or as a suitable reaction product thereof.

The semi-finished products according to the invention, the liquid, semi-solid, and solid end products also contain additives conventional in the art, reference being made to the literature in the field, such as 1, 2 and 3, 4. These additives are emulsifiers, dispersants, moisturizers, fragrances, thickening agents, softeners, preservatives, and substances used for changing the viscosity. The aerosol-like end product compositions also contain stabilizers, propellants, complexes containing aluminiums (perspirants), fragrances and bactericidal substances, which are conventionally used in skin and hair care compositions.

The skin or hair care compositions (=the end product compositions) obtained from the semi-finished products according to the invention contain 0.1 to 20% by weight of the active ingredient mixture according to the invention obtained from wood material, when calculated from the total weight of the product, preferably 0.1 to 10, more preferably 0.5 to 5% by weight, and most preferably 1 to 3% of the active ingredient mixture. Such a large amount of raw product from the total weight of the product is made possible by virtue of the good availability of the raw product and the cost-effective manufacturing method. In addition, the raw product is a physiologically well-tolerated natural product, whereby it can be used in large amounts.

The quality and the proportion of the lignan and flavonoid compounds contained in the raw products according to the invention from the raw product at each time are designed on the basis of the end use. The lignan and flavonoid compounds according to the invention are used in the end products primarily because of their properties to capture free radicals, whereby the selection and the extraction method of the raw wood material are extremely significant to the amount and composition of the active ingredients, i.e., the tannin, lignan, and flavonoid compounds contained in the obtained extract solution or concentrate. The microbicidal properties of the lignan or flavonoid mixture contained in the raw product can also be used as one selection criterion. The active ingredient mixture according to the invention, originating in the raw products, is used in the end products in amounts of 0.2 to 20% by weight, preferably 0.2 to 3% by weight, and more preferably 0.2 to 1% by weight from the total weight of the composition.

The knot wood of spruces and hemlock and pine contain a lot of lignan compounds that mostly contain hydroxymatairesinol or matairesinol, secoisolariciresinol, cyclolariciresinol, lariciresinol, pinoresinol, pinosylvine, and lesser amounts of other lignans and oligolignans. Both hydroxymatairesinol and secoisolariciresinol and their derivatives, such as oxomatairesinol, are relatively good in capturing the free radicals. However, even better capturers of free radicals have proven to be the active ingredient mixtures obtained from the acacia tree, containing melacasidine and teracasidine flavonoids and biflavonoids as their chief components, and the active ingredient mixtures obtained from the eucalyptus tree, which contain ellagatannin. One advantage of the raw products obtained from pine is also the fact that they contain microbicides, improving the preservability of the cosmetic semi-finished products and end product compositions manufactured from these raw products. Example 1 hereinafter describes in detail the varoius lignan, flavonoid and tannin compounds and mixtures of the compounds, which are contained in the raw products according to the invention.

In the following, some new semi-finished products according to the invention and the end product compositions containing the tannin, lignan, and flavonoid mixtures originating in these semi-finished products are described in detail.

Some components of the organic UV protectors penetrate inside the skin. If these components are unstable, they may, when exposed to light, produce free radicals that may induce various skin diseases. Exposure to light can occur, when the sun protection composition wears away from the surface of the skin. The active ingredient mixture is physiologically well-tolerated, non-cytotoxic, whereby it can be used in large amounts in the end product compositions. The large amount of active ingredient mixture in the end composition enables the prevention of the adverse effects of free radicals on the skin, which are generated by the exposure to light of the components of the UV protectors. The good physiological tolerability of the raw product according to the invention also enables novel sun protection compositions that use no UV filter media at all, their protection being merely based on the properties of the active ingredient mixture to capture free radicals. Accordingly, the invention also relates to the manufacture of sun protection compositions that have no actual substances that would filter (absorb) UV radiation, but only the active ingredient mixture that originates in the raw product obtained from wood.

Another important method of implementation of the invention comprises the use of the raw extract or raw liquid, which is obtained from the wood material and contains active ingredients, in protecting coated physical filter particles, such as fine-grained TiO₂ and Zno particles, which are coated with Al and silica compounds and used in sun protection products. The filter particles are conventionally coated with aluminium oxide or sodium silicate and suitable hydrophobic or hydrophilic compounds to render these particles chemically inert. However, the thickness of the coating may vary in places, whereby the filter particles may react with the skin or different parts of the organism, if they have penetrated deeper into the skin/organism. By coating the filter particles with the substance containing the active ingredient mixture, the entry of the free radicals, which are generated by the contact of the physical filter particles and the skin or organism, inside the skin/organism is prevented. In that case, the semi-finished product is obtained by bringing the raw extract, raw extract concentrate or raw liquid containing the active ingredient mixture in contact with the filter particles, and the liquid is evaporated, whereby the active ingredient mixture remains on the surface of the filter particles. Other possibly usable capturers of free radicals comprise pure HMR potassium acetate (a compound containing almost nothing but hydroxymatairesinol), which can be used alone or together with other antioxidants.

One important embodiment of the invention relates to the manufacture of micro capsules, which have a delayed-release form (the so-called depot effect) and contain the raw powder according to the invention or a powder-form semi-finished product made of the raw powder, the micro capsules being incorporated into the desired skin or hair care composition. The microencapsulation is performed by a method conventional as such by coating a core, which contains the effective powdery raw product or its derivative, by a suitable polymer matrix. The polymer matrix is brought onto the core, for example, by spray drying, an air suspension method or polymerizing the polymer matrix onto the core in-situ. The polymer matrix can be manufactured of, for example, silicone polymers, gelatine, polyamide, polymethyl acrylate (PMA), ethylene vinyl acetate (EVA). The coating of the dispersions and suspensions of the liquids and liquids and solid matters according to the invention can be performed by centrifugal extrusion, for example. Regarding the detailed performance of the microencapsulation methods, reference is made to the techniques well-known in the art.

When so desired, the liquid raw extract, raw extract concentrate, and raw liquid obtained from the wood material can also be encapsulated to form liposomal compositions by encasing them in liposomes, such as phospholipids. The liquid semi-finished products made of the raw extract, raw extract concentrate, and raw liquid, such as homogeneous solutions, emulsions, dispersions, and suspensions, which have been dealt with above in connection with the semi-finished products, can be encapsulated to form liposomal compositions. Regarding the detailed performance of the manufacture of the liposomal compositions, reference is made to the techniques well-known in the art, such as 3.

The raw product according to the invention can also be used in the manufacture of foams, powders, and creams to protect operation scars.

### EXAMPLES

### Example 1

### Raw Extracts

The knot wood of the trunk of a full-grown acacia tree (Acacia crassicarpa, Acasia mangium), eucalyptus tree (Eucalyptus globulus) or various fir-trees or conifer trees (Abies balamea, Abies sibrica, Pinus sibrica, Abies pectinata, Pinus sylvestris, Picea abies, Picea pungens (Colorado spruce) was chopped, cool-dried, ground, and extracted into a mixture of butylene glycol and alcohol (butylene glycol/ethanol) or a mixture of propylene glycol and alcohol (propylene glycol/ethanol) or glycerine. The ethanol amine/formic acid ancat solution was also used as extraction solution. Depending of the solution used for the extraction, the extraction solutions comprised an active ingredient mixture containing 7 to 10% by weight of lignans.

The active ingredient mixture content in the raw extracts:

The raw extracts of the knot wood of the acacia tree trunk contained plenty of melacasidine and teracasidine (flavonoids) and also biflavonoids.

The raw extract from the knot wood of the fir tree trunks of the genus Abies contained plenty of lignans (33 to 32%), the main components of which were secoisolariciresinol and lariciresinol.

The raw extracts of conifer trees like spruce and pine contained plenty of lignans (25 to 55%). Hydroxymatairesinol was the prevailing lignan in the spruces of the species Picea.

The amount of lignans in common Norway spruce (Picea abies) was about 50%, the majority being hydroxymatairesinol (60 to 70%) and the rest secoisolariciresinol, a-conidendrine and different oligolignans. If the extraction was carried out with a heated extraction solution (about 200° C.), part of the 7-hydroxymatairesinol changed into 7-oxomatairesinol, its ability to capture free radicals being even better than that of hydroxymatairesinol.

Generally speaking, the antioxidative effect of the raw extracts obtained from the spruces was good, in the same order as the raw extract obtained from the acacia trees; instead, their ability to capture free radicals was weaker.

However, spruce is used to a larger extent than other types of wood in the paper and pulp industry, whereby there are large amounts of raw wood material available, whereby the raw extracts obtained from spruce are competitive with other raw extracts as to their costs, even though their ability to capture free radicals is lower.

The extracts of pine trees (Pinus) also contained plenty of lignans, their abilities to capture free radicals being comparable to Trolox. The lignans of pine trees (Pinus) contained plenty of secoisolariciresinol, lariciresinol, pinosylvine, and nortrachelogenin. In addition, the raw extracts of the conifer trees contained lesser amounts of various unidentified oligolignans, flavonoids, pinodendrine, conidendrine, stilbenes, pinocembrine, and their derivatives.

For example, the raw extract of Pinus sylvestris (Scottish pine) contained a monoether of pinosylvine, nortrachelogenin, and secoisolariciresinol, about 60 to 80% in total, and resin acids. Even though the ability of the raw extract of Pinus Sylvestris to capture free radicals (cf. Table 1) was lower than that of the reference compound (Trolox), it was observed to have antimicrobial effects, making the extract especially interesting for cosmetic products. Compounds having antimicrobial effects were not identified.

The lignans and the flavonoids occurred in the raw products according to the invention as such or as various derivatives and stereoisomeric conformations due to either the manufacturing method of the raw product or the natural isomerism of the lignans and flavonoids contained in the raw wood material and, thus, also the raw extract.

Generally, the derivatives of lignans either naturally occurring in the wood material or generated, when processing the raw products according to the invention include: for hydroxymatairesinol, among others: dimethylmatairesinol, 7-methoxymatairesinol, 7-oxomatairesinol, didemethylmatairesinol, isohydroxymatairesinol, enterolactone, conidendrine, 7′,8′-dehydro-7-hydroxymatairesinol; for the secolariciresinol and lariciresinol that are commonly found in pine and spruce: cyclolariciresinol, 7-hydroxysecoisolariciresinol; for the pinosylvine common in pine: the monoether of pinosylvine, such as monomethylether and dihydropinosylvine and the monomethylester of dihydrosylvine; for pinoresinol: dimethylpinoresinol; for nortrachelogenin: liovil.

The raw products according to the invention contain lignans' hydroxymatairesinol, matairesinol, nortrachelogenin, pinoresinol, secoisolariciresinol, conidendrine; as derivatives, their ethers and esters, ketones, aldehydes, hydrated and dehydrated forms, methylated and demethylated forms, as well as various stereoisomeric conformations. All of these are considered to fall within the scope of the raw products according to the invention. The method of treatment of the raw products, when used in cosmetic end compositions and intermediate products, is performed on the basis of the use, the properties of the raw product to capture free radicals, and possible additional properties, such as antimicrobial properties.

TABLE 1
The ability of some active ingredient extracts and individual
lignan or flavonoid compounds to capture free radicals, compared with
Trolox (6). The extracts originated in the knot wood of the tree trunk.
Capture of peroxyl radicals in vitro, g/mol

| | |
|---|---|
| acacia crassicarpa (raw extract) | 47 |
| melacasidine | 50 |
| cyclolariciresinol | 118 |
| trolox | 125 |
| Eucalyptus globukus (raw extract) | 129 |
| Picea glauca (raw extract) | 129 |
| pinoresinol | 129 |
| teracasidine | 128 |
| Abies pectinata (raw extract) | 147 |
| Acasia mangium (raw extract) | 147 |
| nortrachelogenin | 169 |
| Picea mariana (raw extract) | 172 |
| hydroxymatairesinol | 179 |
| Pinus sylvestris (raw extract) | 206 |
| matairesinol | 345 |
| Abies lasiocarpa (raw extract) | 368 |
| lariciresinol | 370 |

Furthermore, it was discovered that the ability of Picea abies (Norway spruce) to capture peroxyl radicals was about half of that of trolox.

The raw extracts were used in various compositions according to the invention so that the content of the active ingredient was 1 to 5% by weight from the total weight of the composition or the semi-finished product. In the following, some end compositions and semi-finished products according to the invention are illustrated, containing an active ingredient mixture obtained from a raw product originating in branches or the bases of branches.

### Example 2

Sunscreen lotion
Percentages given in percents by weight

| water | | added to 100% |
|---|---|---|
| raw extract based on | | |
| butylene glycol | moistener, solvent | 5% |
| | antioxidant and | |
| | capturer of | |
| | free radicals | |
| xanthan gum | thickening agent | 0.50% |
| chlorphenasin | antibacterial agent | 0.10% |
| aqua, sodium phytate | antioxidant | 0.10% |
| Lecisun 45T | see below | 5-30% |
| isononyl isononanoate | | 10.00% |
| phenonip | | 0.50% |

Lecisun 45T contained 45 to 50% of physical and/or organic sun protection agents, such as TiO₂ or TiO₂ and ZnO and MBBT; as a dispersible oil, isononyl isononanoate 25 to 30%, and 25% of a lamellar phospholipid-based emulsifier (Amisol® Soft). The butylene glycol raw extract mixture contained 10% of a lignan mixture obtained form Norway spruce, and the rest was butylene glycol. Thus, the total content of raw extract in the sunscreen lotion increased to about 0.5% by weight. The raw extract contained hydroxymatairesinol as its main component, and also lesser but, nevertheless, observable amounts of lariciresinol, conidendrine and oligolignans.

What is noteworthy in the sunscreen lotion composition that protects against the UVA/UVB rays of the sun described above, is that the HMR raw extract according to the invention was added to the solvent (butylene glycol) conventionally used as solvent, in an amount that protects the skin antioxidatively and by possibly capturing the free radicals generated from the sunscreen lotion itself by means of light from penetrating deeper into the skin. In this way, it replaces the BHA or BHT (butylated hydroxy aminol, butylated hydroxy toluene), which is normally used as the antioxidant, both of them being cytotoxic and, thus, ill-tolerated, whereby their quantities of use must be kept low (about 0.05% by volume). When the antioxidant is used in too small quantities, its effectiveness is questionable. Instead, because of its non-toxicity, the raw extract according to the invention can be used in large enough amounts, whereby its antioxidative effect and the ability to capture free radicals are adequate.

### Example 3

### Gel

A gel that protects against the UVA/UVB light of the sun, wherein the raw extract containing the active ingredient mixture was used in an amount of 5% by weight, mixed with glycerine. The amount of active ingredient mixture was then about 0.3% from the total weight of the end composition. All percentages are given in percents by weight.

Component A

| | | |
|---|---|---|
| deionized water | solvent | up to 100% |
| chlorphenasin | antibacterial agent | 0.10 |
| aqua, sodium phytate | antioxidant | 0.10 |
| raw extract + glycerine | moistener, solvent + | 6.00 |
| | capturer of free | |
| | radicals and antioxidant | |

Component B

| | | |
|---|---|---|
| gelling agent | | 3.00 |
| cyclomethicone | oil-based solvent | 2.00 |
| butylene glycolide caprylate/ | retainer of dispersion | 4.00 |
| dicaprate | | |
| cetaryl isononanoate | skin protector | 2.00 |
| avobenzone | UVA protector | 2.00 |
| polysilicone 15 | UVB protector | 2.00 |
| diethylamino hydroxybenzoyle | UVA protector | 2.00 |
| hexyl benzoate | | |
| octocrylene | softener | 4.00 |
| phenoxy ethanol, paraben esters | preservative | 0.50 |

Manufacture: The aqueous component A and the oily component B are mixed together, homogenized, and the pH is adjusted at room temperature.

The sodium phytate can also often be omitted from this gel, or decrease its amount, as the glycerine-based raw extract can be used in sufficiently large amounts to ensure the antioxidative ability and the capacity to capture free radicals. The active ingredient mixture contained in the raw extract originated in the knot wood of the trunks of either spruce or acacia. If the raw extract obtained from the knot wood of pine trunks is used, the ability of which to capture free radicals is slightly weaker than that of the HMR raw extract of spruce trunks, but which has antimicrobial properties, the phenacin can be fully or partly omitted and increase the amount of glycerine+raw extract in the formula.

### Example 4

Sunscreen composition
Component A

| | | |
|---|---|---|
| deionized water | solvent | up to 100% |
| chlorphenasin | antibacterial agent | 0.10 |
| aqua, sodium phytate | antioxidant | 0.10 |
| xanthan gum | thickening agent | 0.50 |
| Raw extract: butylene glycol + | solvent, moistener, | 10.00 |
| active ingredient mixture | antioxidant, capturer | |
| | of free radicals | |

Component B

| | | |
|---|---|---|
| Granlux TEM 55 M1 | see below | 20.00 |
| isononyl isononanoate | dispersing oil | 3.00 |
| avobenzone | UVA protector | 4.00 |
| polysilicone 15 | UVB protector | 2.00 |
| butylene glycolide caprylate/ | oil-based solvent | 2.00 |
| dicaprate | | |
| phenoxy ethanol, paraben ester | preservative | 0.50 |

Manufacture: The aqueous component A and the oily component B are mixed together, homogenized, and the pH is adjusted at room temperature.

Granlux TEM 55 M1 contained a physical sun-protector, softener, viscosity adjuster, emulsion retainer, and vegetable-based emulsifier.

The raw extract originated in the knot wood of either spruce, eucalyptus or acacia. The amount of active ingredient mixture in the raw extract was about 10% from that of butylene glycol, whereby in the finished composition, the total amount of active ingredient mixture originating in the raw extract was about 1% by weight. By using the raw extract, it was possible to entirely replace the BHT, which at present has proven to be cytotoxic.

### Example 5

O/W sunscreen lotion
Component A

| deionized water | solvent | up to 100% |
|---|---|---|
| chlorphenasin | antibacterial agent | 0.10 |
| aqua, sodium phytate | antioxidant | 0.10 |
| xanthan gum | thickening agent | 0.50 |
| butylene glycol | solvent, moistener, | 4.50 |

Component B

| | | |
|---|---|---|
| TG2 50 M1 + active ingredient | see below | 30 |
| mixture | | |
| cetaryl isononanoate | skin protector | 2.00 |
| phenoxy ethanol, paraben ester | preservative | 0.50 |
| stearyl heptanoate | softener | 3.00 |
| dimethicone | softener | 1.00 |
| cyclomethicone and | softener | 1.00 |
| dimethiconale | | |

The aqueous phase A and the oily phase B were mixed together heating at 75 to 80° C., and homogenized, the pH was adjusted at room temperature.

TG2 50 M1+active ingredient mixture contains:
TiO₂ as the physical UV protector (UV-filter).
Caprylic/capric acid glycerides, with which a butylene glycol-based raw extract was blended. This mixture was used as the dispersing agent and the softener, to which the active ingredient mixture (HMR lignan mixture) according to the invention, which had been isolated from spruce, gave antioxidative properties and those enhancing the capture of free radicals. The end product contained about 2.5% by weight of lignan mixture (mainly hydroxymatairesinol).
Polyhydroxystearic acid to retain the emulsion
Cross-linked polymethyl acrylate as the thickening agent.

### Example 6

### Solid Dispersion as a Sunscreen Composition

The raw extracts or ground knot wood powder can be used in the solid dispersions containing a UV protector, which are disclosed in U.S. Pat. No. 5,811,082 and which are formed by dispersing the UV protector particles in wax or “ancat” waxes that contain “ancat” compounds, which waxes are solid at room temperature but liquid at an increased temperature. The ancat compound is a surface-active amphoteric-like substance, which is formed by mixing anionic and cationic components with each other. In that case, the ground raw powder obtained from the knot wood of the tree trunk can be mixed together with the solid UV protector particles in the molten wax phase. Then, the raw extract according to the invention, in turn, is preferably either mixed with the emulsifier or the dispersant used in the publication or with a mixture of anionic and cationic surfactants and polymers, such as the “Ancat 1” (dodecyl methyl ammonium/dodecyl sulphate) or “Ancat 2” mixtures (laurinic acid diethyl ether/dodecylamine) cited in the publication. When manufacturing the ancat waxes, the ancat components are dissolved separately in an aqueous medium, and the solutions and the raw extract according to the invention are mixed with each other to form an aqueous mixture.

The solid wax dispersions thus obtained, which contain antioxidative components/those that capture free radicals and originate in the raw extract or the raw powder according to the invention, UV protectors, and possible additives, can be used as cosmetic preparations as such or, alternatively, they can be blended to be part of the cosmetic preparations that are manufactured. Regarding the details of the manufacturing method of the solid wax dispersions mentioned above, and the more detailed compositions and use of the various cosmetic products and semi-finished products thus obtained, reference is made to the US patent mentioned above.

### Example 7

### Colouring Agent Similar to Melanin (Semi-Finished Product)

The concentrated raw extract according to the invention was added to Granlux® Melanin Mimic™ TB concentrate containing a UV protector, which is supplied by Granula Oy. This emulsion concentrate contained the UV protector, iron oxide, various emulsion retainers (cetaryl alcohol, cetaryl glucoside, polyhydroxy stearic acid); as the dispersant, PEG 40 esters of apricot kernel oil; aluminium and dimethicone as viscosity adjusters, a softener (hydrated decene oligomers).

The emulsion thus obtained can be used in the manufacture of various end products of colour cosmetics that require UV protection, such as lipsticks and the sprays used in hair cosmetic products.

### Example 8

### After-Shave Gel

Shaving products do not require UVA or UVB protectors but, instead, the presence of capturers of free radicals is desirable. This prevents the free radicals induced from the components of the products from getting in contact with the skin.

### Phase A

carbomer
water

### Phase B

PEG-40 hydrated castor oil
menthol
panthenol, propylene glycol
raw extract: triethanol amine+alcohol+active ingredient mixture
benzophenone-2
water
C.I. 42051, Acid blue 3
Manufacture: Phase A is allowed to expand and Phase B is added thereto. In Phase B, the raw extract was manufactured by extracting the wood chips obtained from knot wood with a solvent that contained triethanolamine and ethanol. The disclosed shaving product contained over 1% by weight of the active ingredient mixture (HMR raw extract of spruce).

### Example 9

### After-Sun Spray

Sprays are popular ways of dosing cosmetic products. The after-sun sprays need no UVA or UVB protectors and the active ingredient mixtures according to the invention function in them by preventing the effect of the free radicals induced by the thermal stress on the skin (they are generated from the components of the after-sun products). As the product is not exposed to UV radiation, the amount of active ingredient mixture can be kept at 1% by weight.

% by weight

Phase A

| | |
|---|---|
| deionized water | 84 |
| hydroxyethyl cetyl moniumphosphate | 2.00 |
| D panthenol | 0.5 |
| dimethicone copolyol | 0.5 |
| raw extract (propylene glycol + active ingredient mixture) | 10 |

Phase B

| | |
|---|---|
| PEG-40 hydrated castor oil | 0.3 |
| fragrance | |

In the end product composition, the raw extract contained about 10% of active ingredient mixture, whereby the amount of active ingredient mixture in the end product was then about 1% by weight.

### Example 10

Lipstick

| | |
|---|---|
| ingredient | % by weight |

A

| | |
|---|---|
| Granlux CCA-50 + active ingredient mixture | 9.00 |
| butyl stearate | 23.00 |
| Ricinus communis | 43.50 |

A1

| | |
|---|---|
| C177491 + C177492 (colouring agent) | 3.5 |
| titanium dioxide | 3.5 |
| CL 15850 (colouring agent) | 1.00 |

B

| | |
|---|---|
| ozokerite | 4.00 |
| candelila wax | 5.00 |
| topopherol acetate | 0.50 |
| C10-30 cholesterol/lanosterol ester | 2.00 |
| Cera alba | 5.00 |

C

| | |
|---|---|
| perfume | 0.25 |

Phases A, A1, and B are mixed with each other homogeneously, heating at the same time. The perfume is added and the composition is placed in a mould and cooled.

Granlux CCA-50 contained titanium dioxide, beeswax, Carnauba, dimethicone, stearic acid, aluminium, and the active ingredient mixture according to the invention, obtained from spruce, as the capturer of free radicals. CCA-50 is an intermediate product according to the invention, wherein the liquid raw extract obtained from the knot wood of spruce trunk is blended with an aluminium compound. In that case, the possible thinner spots of coating in the aluminium coating of the fine-grained titanium dioxide or the lack of the entire coating does not cause problems, as the free radicals induced by the titanium dioxide particles in a possible skin contact or when entering the organism cannot damage the skin/organism.

### Example 11

Sunscreen lotion

| | |
|---|---|
| ingredient | % by weight |

Component A

| | |
|---|---|
| carbomer | 15 |
| EDTA | 0.05 |
| water | 24.60 |

Component B

| | |
|---|---|
| benzophenone-3 | 4.00 |
| cetyl dimethicone copolyol | 2.00 |
| cyclomethicone | 3.00 |
| diethyl toluamide | 20.00 |
| dimethicone | 2.00 |
| glyceryl stearate, PEG-100 stearate | 2.00 |
| octyl salicylate | 5.00 |
| octyl methyl cinnamate | 7.50 |

Component C

| | |
|---|---|
| trimethyl siloxy silicate | 1.00 |

Component D

| | |
|---|---|
| HMR raw extract: Triethanol amine + | 10.35 |
| HMR active ingredient mixture | |

E

| | |
|---|---|
| propylene glycol, diazodinyl urea | 0.35 |
| methyl paraben, propyl paraben | |
| perfume | 0.5 |

The ingredients of Component A are mixed together by slowly adding carbomer. Component A is mixed with Component B, heating to 75° C. at the same time. Components C and D are mixed with Component AB at 75° C. Finally, Component F is mixed with Component ABCD and cooled, homogenized and packed. Example 11 is a sunscreen lotion containing an organic UV filter medium (octyl methyl cinnamate, benzophenone), which also includes an insect repelling substance. The amount of lignan mixture originating in the HMR raw extract and mainly containing HMR is 1.35% by weight of the composition. The lignans capture the free radicals, which are induced by light from the components absorbed inside the skin, from getting in contact with the skin. This is necessary especially, when the sunscreen lotion wears or is rinsed off the surface of the skin.

### Example 12

### Concentrate

HMR raw extract was extracted with propylene glycol from the knot wood of Norway spruce (Picea abies) trunks, containing about 10% of lignan mixture (=active ingredient mixture), the main part thereof comprising hydroxymatairesinol. Liquid 3-[(2-ethylhexyl)oxo]-1,2-propanediol concentrate (ethylhexyl glycerine, also known by the trade name Sensiva SC50) was dissolved in the raw extract thus obtained, so that the ratio of the amount of lignan mixture to ethylhexyl glycerine (p/p) varied within 3:1-1:3. The homogeneous mixture thus obtained was concentrated. It was possible to use the semi-finished concentrated product in deodorants and various skin care products as a microbicidal mixture that captures free radicals.

### REFERENCES

1. Die kosmetischen Präparate, 4th edition, Bd. 1, Die Parfumerie, Grundlagen der Kompositiontechnik, Verlag fur chem. Industrie, edited by Andreas Domsch, Augsburg 1992.
2. Die kosmetischen Präparate, 4th edition, Bd. II, Wässrige und tensidhaltige Formulierungen, Verlag fur chem. Industrie, edited by Andreas Domsch, Augsburg 1992.
3. Die kosmetischen Praparate, 4th edition, Bd. III, Lipidhaltige und emulgierte Formulierungen, Verlag fur chem. Industrie, edited by Andreas Domsch, Augsburg 1992.
4. International Nomenclature of Cosmetic Ingredients (INCI).
5. FDA's list of approved cosmetic substances.
6. Pietarinen et al., Kontwood extracts-rich sources of strong antioxidants, Seminar Publication 3. Italian Meeting of lignin Chemistry, 23-24 Jun. 2005.

1. A semi-finished product, which is manufactured from the raw liquid, raw extract or raw powder obtained from wood material, an end product composition for skin or hair care being obtained from the semi-finished product by adding one or more components thereto, characterized in that
the semi-finished product optionally contains an UV protector,
the semi-finished product is a liquid, such as solution, emulsion, microemulsion or nanoemulsion or a semi-solid substance, such as dispersion, suspension, wax, lotion or gel or a solid substance, and the skin or hair care composition (the end product) can be manufactured from the semi-finished product by adding liquid thereto,
the semi-finished product contains an active ingredient mixture, which is obtained from wood material by extracting, pressing or grinding, and which contains phenolic compounds obtained from the wood material, the compounds having antioxidative and/or microbicidal properties and/or abilities to capture free radicals,
the active ingredient mixture contains at least one oligolignan as phenolic compounds,
in addition, the active ingredient mixture contains, as phenolic compounds, at least two lignans, tannins or flavonoids, which are selected from a group consisting of hydroxymatairesinol, matairesinol, oxomatairesinol, lariciresinol, cyclolariciresinol, secoisolariciresinol, nortrachelogenin, pinoresinol, pinosylvine, conidendrine, melacasidine, teracasidine, ellagatannin or a reaction product, stereoisomer or derivative of these compounds.

2. A semi-finished product according to claim 1, characterized in containing 0.1 to 5%, preferably 0.5 to 3% of oligolignans.

3. A semi-finished product according to claim 1, characterized in that the active ingredient mixture contained therein is pressed, extracted or ground from wood material, which originates in knot wood of a tree trunk, branches, bark or birch bark.

4. A semi-finished product according to claim 3, characterized in that the active ingredient mixture is obtained by extracting, pressing or grinding the knot wood, branches, and/or bark of the wood material of at least two different wood species, such as spruce and pine, or conifer and birch.

5. A semi-finished product according to claim 4, characterized in that the active ingredient mixture contains lignans extracted from spruce material and microbicidal compounds extracted from pine.

6. A semi-finished product according to claim 4, characterized in that the active ingredient mixture contains lignans extracted from coniferous material and betulinic acid or a derivative thereof extracted from birch.

7. A semi-finished product according to claim 1, characterized in that other antioxidants and capturers of free radicals, such as vitamins, known as such, have been added thereto.

8. A semi-finished product according to claim 1, characterized in that the raw extract, raw liquid, or raw powder, which is ground, extracted or pressed from the wood material and contains the active ingredient mixture, is used as such as the semi-finished product or a component thereof.

9. A semi-finished product according to claim 1, characterized in that the active ingredient mixture is obtained by extracting the knot wood of chopped trunk material or chopped knot wood material with a physiologically tolerated solvent, such as an organic solvent containing alcohol, or an ancat solvent.

10. A semi-finished product according to claim 9, characterized in that the active ingredient mixture is obtained by extracting the wood material or knot wood of tree trunks with a lower monovalent alkyl alcohol or a mixture of the monovalent lower alkyl alcohol and a bivalent alcohol, or a mixture of the monovalent lower alkyl alcohol and a trivalent lower alkyl alcohol, or an ancat solution mixture, which is a solution with buffer properties that is formed from a solution-form short-chain acidic organic compound and a short-chain solution-form alkaline organic compound.

11. A semi-finished product according to claim 10, characterized in that the monovalent alcohol is ethanol, propanol, butanol, hexanol, heptanol, octanol or decanol, preferably ethanol.

12. A semi-finished product according to claim 10, characterized in that the bivalent alcohol is glycol, propylene glycol, butylene glycol, pentylene glycol or dipropylene glycol, preferably butylene glycol or dipropylene glycol.

13. A semi-finished product according to claim 10, characterized in that the trivalent alcohol is glycerol.

14. A semi-finished product according to claim 1, characterized in that the raw extract includes an active ingredient mixture comprising oligolignans, lignan, tannin and/or flavonoids in an amount of over 7% by weight, preferably over 10% by weight from the total weight of the extract.

15. A semi-finished product according to claim 1, characterized in that the active ingredient mixture contained therein is a solution-like HMR raw product containing oligolignans extracted from the knot wood or the branches of conifer tree trunks, and lignans that contain about 60 to 70% of hydroxymatairesinol, matairesinol and/or oxomatairesinol.

16. A semi-finished product according to claim 15, characterized in that the lignans of the active ingredient mixture in the HMR raw product include hydroxymatairesinol and/or matairesinol and/or oxomatairesinol and secoisolariciresinol and conidendrine, and the oligomers thereof.

17. A semi-finished product according to claim 1, characterized in that the active ingredient mixture added to the semi-finished product contains oligolignans or lignans, which are antioxidative and/or have properties to capture free radicals, to the extent that they alone prevent the formation of free radicals from the substance used as the UV filter in the semi-finished product and/or in the end product manufactured from the semi-finished product.

18. A semi-finished product according to claim 17, characterized in that the active ingredient mixture added to the semi-finished product contains oligolignans or lignans, which are antioxidative and/or have properties to capture free radicals, to the extent that they prevent the formation of free radicals from a substance that has come off the colour cosmetic composition, such as a lipstick or lipsalve, which is manufactured form the semi-finished product, when these substances come in contact with the organism.

19. A method of producing an end product composition, comprising adding at least a solvent to the semi-finished product according to claim 1, wherein the semi-finished product is a liquid-liquid emulsion dispersion concentrate or liquid-liquid emulsion suspension concentrate, microemulsion concentrate or nanoemulsion concentrate that possibly contains a UV protector, such as TiO₂, FeₓO_{y}, ZnO, or an organic UV filter medium, such as MBBT, and the end product composition is skin/hair care composition or a component thereof.

20. The method according to claim 19, further comprising adding an emulsifier to the semi-finished product.

21. The method according to claim 19 wherein the end product composition contains physical UV filters, such as fine-grained FeₓO_{y}, TiO₂, ZnO and/or CeO₂ particles, on top of which a coating has been deposited, containing silicate or aluminium compounds and also including the active ingredient mixture.

22. An end product comprising the semi-finished product according to claim 1 wherein the in the manufacture of an end product composition is a sunscreen composition containing a UVA and/or UVB filter medium, an after-sun product or an after-shave product that contains no UV filter media, or a colour cosmetic product, such as a lipsalve or a lipstick that possibly contains UV filtering media.

23. An end product comprising the semi-finished product according to claim 1 wherein the end product composition is a substance similar to melanin, the active ingredient mixture therein containing compounds that capture free radicals in an amount sufficient to prevent the components of the end product produced from the semi-finished product from oxidizing, when coming in contact with the skin, a UV protector and a colouring agent.

24. An end product comprising the semi-finished product according to claim 1 wherein the product composition is intended to protect the hair against the sun and contains 0.5 to 5% of the active ingredient mixture and UVA and/or UVB protectors (UV filters).

25. A semi-finished product according to claim 17, characterized in containing at least 5%, preferably at least 10% of lignans or oligolignans that have antioxidative properties and/or abilities to capture free radicals, and that the end product contains at least 0.5%, preferably at least 1% of lignans or oligolignans that are antioxidative and/or have abilities to capture free radicals.

26. A semi-finished product according to claim 1, characterized in that the end product contains 0.1 to 20% by weight of the active ingredient mixture, when calculated from the total weight of the product; preferably 0.3 to 5% by weight, and more preferably 0.3 to 1% of the active ingredient mixture from the total weight of the product.

27. A semi-finished product according to claim 1, characterized in containing a non-polar solvent originating in the raw extract of raw extract concentrate, the active ingredient mixture, one or more dispersants, possibly a polar solvent, and possibly additives conventional in the cosmetic field.

28. A semi-finished product according to claim 1, characterized in containing a non-polar solvent, such as glycerol or glycerol alcohol, originating in the raw extract or raw extract concentrate, a UV protector and, possibly, substances, such as emulsifiers and/or dispersants that facilitate the processing of the semi-finished product in forming the end composition, and possibly other additives conventional in the cosmetic field, such as moisteners, thickening agents, moisture-absorbing agents, softeners, lubricants and/or fragrances.

29. A semi-finished product according to claim 28, characterized in being an emulsion dispersion or emulsion suspension or a homogeneous solution, and also containing a surfactant that facilitates the further processing of the semi-finished product and is selected from a group consisting of lecithin and its derivatives, long-chain alkyl amines, and alkyl ammonium compounds, polyethoxylated amines and pyridium salts, capryl/caprine mono/diglyceride, a mixture of alkyl glycoside and alkyl alcohol, polyglycerol-3-di-isostearate and polyglycerol+dihydroxystearate, such as Lameform TGI/Dehumuls PGPH, cetyl polyglyceride PEG/PPI dimethicone, polyglycerol-4-isostearate+cetyl dimethicone copolyol, silicone-based emulsifiers, polyglycerol-isostearate+polyglyceryl-3-methylglucosedistearate the mono/diglycerin of kapryl/kaprine, a mixture of alkyl glycoside and alkyl alcohol, polyglycerol-3-di-isostearate or polyglycerol+dihydroxystearate, such as Lameform TGI/Dehumuls PGPH.

30. A semi-finished product according to claim 29, characterized in being a homogeneous solution containing a fragrance, which is selected from a group consisting of phenyl ethyl glycol, eugenol, isoeugenol, geraniol, citronellol or linalol or their esterified forms or their aldehydes.

31. A semi-finished product according to claim 1, characterized in that the composition of the active ingredient mixture is selected on the basis of the polarity of the application.

32. The semi-finished product according to claim 1, wherein the active ingredient mixture, which is obtained from wood material by extracting, pressing or grinding, and which contains phenolic compounds obtained from the wood material, the compounds having antioxidative and/or microbicidal properties and/or abilities to capture free radicals, the active ingredient mixture also containing as phenolic compounds at least one oligolignan, and also containing as phenolic compounds at least two lignans, tannins or flavonoids selected from a group consisting of hydroxymatairesinol, matairesinol, oxomatairesinol, lariciresinol, cyclolariciresinol, secoisolariciresinol nortrachelogenin, pinoresinol, pinosylvine, conidendrine, melacasidine, teracasidine, ellagatannin or a reaction product, stereoisomer or derivative of these compounds.
